(19) United States
(12) Patent Application Publication
(10) Pub. No.: US 2022/0395477 A1
(43) Pub. Date: Dec. 15, 2022

(54) COMPOSITIONS AND METHODS FOR PREVENTING AND TREATING RESPIRATORY SYNCYTIAL VIRUS INFECTION

(71) Applicants: Shyam S. MOHAPATRA, Lutz, FL (US); Subhra MOHAPATRA, Lutz, FL (US); Andrew Robert MCGILL, Tampa, FL (US); Eleni MARKOUTSA, Tampa, FL (US)

(72) Inventors: Shyam S. MOHAPATRA, Lutz, FL (US); Subhra MOHAPATRA, Lutz, FL (US); Andrew Robert MCGILL, Tampa, FL (US); Eleni MARKOUTSA, Tampa, FL (US)

(21) Appl. No.: 17/773,257

(22) PCT Filed: Oct. 29, 2020
(86) PCT No.: PCT/US2020/057897

§ 371 Date: Apr. 29, 2022

Related U.S. Application Data
(60) Provisional application No. 62/927,489, filed on Oct. 29, 2019.

(51) Int. Cl. A61K 31/19

(52) U.S. Cl. CPC A61K 31/19

(57) ABSTRACT

The present disclosure relates to compositions and uses thereof for preventing and treating respiratory syncytial vims (RSV) infection. In some aspects, disclosed herein is a method for preventing or treating respiratory syncytial vims (RSV) infection in a subject, comprising administering to the subject a therapeutically effective amount of a composition comprising a short-chain fatty acid.

### CROSS-REFERENCE TO RELATED APPLICATIONS

This application claims the benefit of U.S. Provisional Application No. 62/927,489, filed Oct. 29, 2019, which is expressly incorporated herein by reference.

### STATEMENT REGARDING FEDERALLY SPONSORED RESEARCH

This invention was made with government support under Grant Nos. IK6BX003778, IK6BX004212, and BX003685 awarded by U.S. Department of Veterans Affairs. The government has certain rights in the invention.

### FIELD

The present disclosure relates to compositions and uses thereof for preventing and treating Respiratory Syncytial Virus (RSV) infection.

### BACKGROUND

Human Respiratory Syncytial Virus (RSV) is a major respiratory pathogen and the main causative agent of pediatric bronchiolitis worldwide but is also responsible for increases in pneumonia rates in the elderly and immunocompromised as well. Clinically, RSV presents itself as an upper respiratory tract infection; however, serious complications can arise such as bronchiolitis in young children, pneumonia, apnea, respiratory failure, the development of asthma, and even death. Annually, in the United States, RSV is the leading cause of pediatric hospitalization with an average of 2.1 million outpatient visits among children younger than five years old. Furthermore, there are ˜60,000 hospitalizations for severe pediatric complications and ˜177,000 hospitalizations among adults older than 65 annually. Globally, RSV causes ˜64 million infections and ˜166,000 deaths annually.

The only approved therapies for RSV disease are aerosolized Ribavirin and palivizumab, an anti-F monoclonal antibody therapy used as prophylactic for high-risk groups, such as premature infants. However, these treatments have moderate efficacy and come at a very high financial cost. This illustrates the need for the development of a safe and effective therapy against RSV. A broad range of anti-RSV efforts using either vaccines or passive prophylaxis with fusion and/or replication inhibitors are being developed but have yet to show any proven therapeutic results. What is needed are compositions and methods for preventing and treating RSV infection.

The compositions and methods disclosed herein address these and other needs.

### SUMMARY

In some aspects, disclosed herein is a method for preventing or treating respiratory syncytial virus (RSV) infection in a subject, comprising administering to the subject a therapeutically effective amount of a composition comprising a short-chain fatty acid.

In some embodiments, the short-chain fatty acid is produced by bacteria (e.g., Bacteroidetes and/or Fimicutes). In some embodiments, the short-chain fatty acid is produced by Bacteroidetes or Fimicutes. In some embodiments, the short-chain fatty acid is produced by Bacteroidetes. In some embodiments, the short-chain fatty acid is produced by Fimicutes.

In some embodiments, the short-chain fatty acid comprises acetate, butyrate, or propionate or a combination thereof. In some embodiments, the short-chain fatty acid comprises acetate. In some embodiments, the short-chain fatty acid comprises butyrate. In some embodiments, the short-chain fatty acid comprises propionate.

In some embodiments, the short-chain fatty acid comprises acetate and butyrate. In some embodiments, the short-chain fatty acid comprises acetate and propionate. In some embodiments, the short-chain fatty acid comprises butyrate and propionate.

In some embodiments, the short-chain fatty acid is encapsulated within a nanoparticle.

In some aspects, disclosed herein is a composition for preventing or treating respiratory syncytial virus (RSV) infection, wherein the composition comprises acetate and butyrate, acetate and propionate, or butyrate and propionate.

In some aspects, disclosed herein is a composition for preventing or treating respiratory syncytial virus (RSV) infection, wherein the composition comprises acetate and butyrate.

In some aspects, disclosed herein is a composition for preventing or treating respiratory syncytial virus (RSV) infection, wherein the composition comprises acetate and propionate.

In some aspects, disclosed herein is a composition for preventing or treating respiratory syncytial virus (RSV) infection, wherein the composition comprises butyrate and propionate.

### BRIEF DESCRIPTION OF THE DRAWINGS

The accompanying figures, which are incorporated in and constitute a part of this specification, illustrate several aspects described below.

FIGS. 1A-1C show that RSV infection alters the gut microbiota profile. The fecal material was collected every day during the study period from control, uninfected (FIG. 1A) and Mock-infected (FIG. 1B) and RSV-infected (FIG. 1C) female BALB/c mice. Genomic DNA isolated from the stool using QIAamp DNA stool minikit. Then, 16S rRNA gene-targeted group-specific primers designed and validated for quantification of the predominant bacterial species in mouse feces by real-time PCR.

FIGS. 2A-2C show that treatment of nanosystem that inhibits RSV infection reverses the gut microbiota profile prior to infection. The fecal material was collected every day during the study period from control, mock-infected (FIG. 2A), RSV-infected (FIG. 2B), and RSV infected, anti-ICAM coated nanoparticle-treated (FIG. 2C) female BALB/c mice. Genomic DNA isolated from the stool using QIAamp DNA stool minikit. Then, 16S rRNA gene-targeted group-specific primers designed and validated for quantification of the predominant bacterial species in mouse feces by real-time PCR.

FIGS. 3A-3C show the effects of varying concentrations of SCFAs in their ability to reduce RSV infection in A549 cells using a RSV-A2-L19F strain (3 MOI) variant that expressed red fluorescent protein (RFP). Cells were incubated in OptiMEM media for 48 hours in varying concentrations of the SCFAs (50-400 uM), such as acetate (FIG. 3A), butyrate (FIG. 3B), and propionate (FIG. 3C), then stained with DAPI, and imaged with Keyence. ImageJ was used to analyze and quantify the fluorescence images.

FIGS. 4A-4B show that A549 cells treated with SCFAs reduce expression of mKate2 RFP encoded by rA2-KL19F RSV. FIG. 4A shows fluorescence microscopy of NucBlue stained A549 cells pretreated 24 hours prior to infection with SCFAs at the indicated concentrations, then challenged with RSV. Images taken after 48 Hrs. FIG. 4B shows RFP/DAPI calculations from mean intensity analyzed with ImageJ. N=3. *p=<0.05; **p=<0.01.

FIG. 5 shows that novel prophylactic combinations of SCFAs reduce RSV expression of MKate2 RFP 48 hrs post infection in A549 cells (ImageJ & Keyence Microscopy). N=3. **p=0.01; ***p=0.001.

FIGS. 6A-6B show that 200 μM single SCFA administration (FIG. 6A) and 150 μM prophylactic combination (FIG. 6B) of SCFAs reduce expression of RSV N as determined by qPCR in A549 cells. Expression normalized to GAPDH. N=4. ***p=0.001; ****p=0.0001.

FIGS. 7A-7B show the Qiagen's Ingenuity Pathway Analysis (IPA) for FFAR3 (GPR41) (FIG. 7A) and FFAR2 (GPR43) (FIG. 7B).

FIGS. 8A-8B show that SCFA combination prophylaxis shows a greater increase in IFNB compared to single administration of individual SCFAs in A549 cells. FIG. 8A shows individual administration of 200 μM SCFA, FIG. 8B shows 150 μM combinations of SCFAs, 24 hrs before infection. Data collected 48 hrs after RSV infection. IFNB detected by qPCR. N=3. **p=0.05; ***p=0.001; ****p=0.0001.

### DETAILED DESCRIPTION

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs.

### Terminology

Terms used throughout this application are to be construed with ordinary and typical meaning to those of ordinary skill in the art. However, Applicant desires that the following terms be given the particular definition as defined below.

As used herein, the article “a,” “an,” and “the” means “at least one,” unless the context in which the article is used clearly indicates otherwise.

The terms “about” and “approximately” are defined as being “′close to” as understood by one of ordinary skill in the art. In one non-limiting embodiment, the terms are defined to be within 10%. In another non-limiting embodiment, the terms are defined to be within 5%. In still another non-limiting embodiment, the terms are defined to be within 1%.

“Activate”, “activating”, and “activation” mean to increase an activity, response, condition, or other biological parameter. This may also include, for example, a 10% increase in the activity, response, or condition, as compared to the native or control level. Thus, the increase can be a 10, 20, 30, 40, 50, 60, 70, 80, 90, 100%, or any amount of reduction in between as compared to native or control levels.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and the administration by another.

The term “biocompatible” generally refers to a material and any metabolites or degradation products thereof that are generally non-toxic to the recipient and do not cause significant adverse effects to the subject.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “composition” is intended to include a combination of active agent and another compound or composition, inert or active.

“Decrease” can refer to any change that results in a lower level of gene expression, protein expression, amount of a symptom, disease, composition, condition, or activity. A substance is also understood to decrease the level of the gene, the protein, the composition, or the amount of the condition when the level of the gene, the protein, the composition, or the amount of the condition is less/lower relative to the output of the level of the gene, the protein, the composition, or the amount of the condition without the substance. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Increase” can refer to any change that results in a higher level of gene expression, protein expression, amount of a symptom, disease, composition, condition, or activity. A substance is also understood to increase the level of the gene, the protein, the composition, or the amount of the condition when the level of the gene, the protein, the composition, or the amount of the condition is more/higher relative to the output of the level of the gene, the protein, the composition, or the amount of the condition without the substance. Also, for example, an increase can be a change in the symptoms of a disorder such that the symptoms are less than previously observed. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

The term “subject” refers to, for example, a human in need of treatment for any purpose, and more preferably a human in need of treatment to treat a disease or disorder. The term “subject” can also refer to non-human animals, such as dogs, cats, horses, cows, pigs, sheep and non-human primates, among others.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, Pa., 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone;

amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, N.J.), polyethylene glycol (PEG), and PLURONICS™ (BASF; Florham Park, N.J.). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

As used herein, the terms “treating” or “treatment” of a subject includes the administration of a composition to a subject with the purpose of curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, and improvement or remediation of damage.

“Therapeutically effective amount” or “therapeutically effective dose” of a composition (e.g. a short-chain fatty acid) refers to an amount that is effective to achieve a desired therapeutic result. In some embodiments, a desired therapeutic result is the prevention of an RSV infection and/or a symptom thereof. In some embodiments, a desired therapeutic result is the treatment of an RSV infection and/or a symptom thereof (e.g., a decrease in viral titer, a decrease in viral nucleic acid levels). Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

### Compositions and Methods

RSV infection-induced bronchiolitis in neonates with immature immune system, is often followed by secondary bacterial infections. The maturation of the host's immune system begins at an early age, and it has been indicated that the composition and diversity of the gut microbiota, which in humans stabilizes in the first year of life, critically affects the development and function of the immune system and the risk of RSV bronchiolitis decreases with a healthy microbiome and bacterial metabolic byproducts from this site can have major impacts on host health and tissue homeostasis. A significant amount of the circulating short-chain fatty acids (SCFAs) come from the fermentation of fiber in the large intestine by the resident gut microbiota. These SCFAs act at the tissue level, promoting barrier integrity and homeostasis, and can systemically promote the development of an antiviral phenotype. SCFAs have the ability to work on G-protein coupled receptors, such as Gpr43 and Gpr41, which can result in activation of interferon. Metabolic profiling of Lactobaccilus johnsonii supplemented mice showed that docosahexaeneoate (DHA) had altered dendritic cells upon RSV infection.

Disclosed herein is a novel combination of metabolites that can be used for treatment and prevention of RSV infection. Accordingly, disclosed herein is a composition for preventing and/or treating respiratory syncytial virus (RSV) infection, wherein the composition comprises one or more fatty acids (e.g., short-chain fatty acids or medium-chain fatty acids).

The respiratory syncytial virus (RSV), a member of the species orthopneumovirus of the Orthopneumovirus genus, is a syncytial virus that causes respiratory tract infections. RSV has a single stranded negative sense RNA genome which is approximately 15.2 Kb long. RSV has been classified into two groups (group A and group B, or termed as “strain A and strain B” herein) on the basis of genetic and antigenic heterogeneity. The two major glycoprotein on the surface of the RSV virion are the attachment glycoprotein (G) and fusion protein (F). G is involved in attachment of virion to the host cells, and F cause the virion membrane to fuse with cell membrane. In addition, four of the viral genes code for intracellular proteins that are involved in genome transcription, replication, and particle budding, namely N (nucleoprotein), P (phosphoprotein), M (matrix protein), and L (“large” protein, containing the RNA polymerase catalytic motifs).

The term “short-chain fatty acid” or “SCFA” herein refers a fatty acid consisting of one to six carbons. Derived from intestinal microbial fermentation of indigestible foods, SCFAs are the main energy source of colonocytes, making them crucial to gastrointestinal health. In some embodiments, the short-chain fatty acid described herein is produced by Bacteroidetes or Fimicutes. In some embodiments, the short-chain fatty acid described herein is produced by Bacteroidetes. In some embodiments, the short-chain fatty acid described herein is produced by Fimicutes. In some embodiments, the short-chain fatty acid is synthetic.

The SCFAs are well known in the art. In some embodiments, the SCFA is selected from formic acid, acetic acid, propionic acid, butyric acid, isobutyric acid, valeric acid, isovaleric acid, or caproic acid, or salts or esters thereof.

The term “non short-chain fatty acid” refers to a fatty acid consisting of more than seven carbons, for example a medium-chain fatty acid (MCFA). A medium-chain fatty acid consists of, for example, about 7-12 carbons. In some embodiments, the fatty acid described herein comprises a medium-chain fatty acid, such as linoleic acid, lauric acid, and/or oleic acid.

In one embodiment, the SCFA comprises acetate, butyrate, or propionate or a combination thereof. In some embodiments, the short-chain fatty acid comprises acetate. In some embodiments, the short-chain fatty acid comprises butyrate. In some embodiments, the short-chain fatty acid comprises propionate. In some embodiments, the short-chain fatty acid comprises acetate and butyrate. In some embodiments, the short-chain fatty acid comprises acetate and propionate. In some embodiments, the short-chain fatty acid comprises butyrate and propionate.

Disclosed herein is a composition for preventing or treating respiratory syncytial virus (RSV) infection, wherein the composition comprises acetate and butyrate, acetate and propionate, or butyrate and propionate.

Also disclosed herein is a composition for preventing or treating respiratory syncytial virus (RSV) infection, wherein the composition comprises acetate and butyrate.

Also disclosed herein is a composition for preventing or treating respiratory syncytial virus (RSV) infection, wherein the composition comprises acetate and propionate.

Also disclosed herein is a composition for preventing or treating respiratory syncytial virus (RSV) infection, wherein the composition comprises butyrate and propionate.

Also disclosed herein is a method for preventing or treating respiratory syncytial virus (RSV) infection in a subject, comprising administering to the subject a therapeutically effective amount of a composition comprising a short-chain fatty acid. In some embodiments, the short-chain fatty acid is produced by Bacteroidetes or Fimicutes. In some embodiments, the short-chain fatty acid is produced by Bacteroidetes. In some embodiments, the short-chain fatty acid is produced by Fimicutes. In some embodiments, the short-chain fatty acid comprises acetate, butyrate, or propionate or a combination thereof. In some embodiments, the short-chain fatty acid comprises acetate. In some embodiments, the short-chain fatty acid comprises butyrate. In some embodiments, the short-chain fatty acid comprises propionate. In some embodiments, the short-chain fatty acid comprises acetate and butyrate. In some embodiments, the short-chain fatty acid comprises acetate and propionate. In some embodiments, the short-chain fatty acid comprises butyrate and propionate. In some embodiments, Bacteroidetes produce acetate and propionate, whereas Firmicutes produce butyrate.

The present invention relates to SCFAs or non short-chain fatty acids in various forms, including SCFA-derivatives, SCFA-pro-drugs, non short-chain fatty acid derivatives, or non short-chain fatty acid-pro-drugs. The preferred SCFA of the present invention, including acetate, propionate, butyrate, may be derivatized to enable modified behavior of the SCFA compound with respect to in vivo half-life, packaging efficiency, production, modified taste or smell.

In some embodiments, the fatty acid (SCFA or non short-chain fatty acids) disclosed herein is encapsulated inside a nanoparticle. The term “nanoparticle” as used herein refers to a particle or structure which is biocompatible with and sufficiently resistant to chemical and/or physical destruction by the environment of such use so that a sufficient number of the nanoparticles remain substantially intact after delivery to the site of application or treatment and whose size is in the nanometer range. For the purposes of the present disclosure, a nanoparticle typically ranges between from about 1 nm to about 1000 nm, or from between about 50 nm and about 500 nm, or from between about 50 nm and about 350 nm, or from between about 100 nm and about 350 nm, between about 120 nm and about 320 nm, between about 140 nm and about 300 nm, between 150 nm and about 280 nm, between 160 nm and about 250 nm, or about 150 nm, about 160 nm, about 170 nm, about 180 nm, about 190 nm, about 200 nm, about 210 nm, about 220 nm, about 230 nm, about 240 nm, about 250 nm, about 300 nm, about 400 nm, about 500 nm, about 600 nm, about 700 nm, about 800 nm, about 900 nm, or about 1000 nm.

The amount of fatty acid (SCFA or non short-chain fatty acids) that can be present in the nanoparticle can be from about 0.1% to about 90% of its nanoparticle weight. For example, the amount of SCFA present in the nanoparticle can be from about 0.1%, about 0.2%, about 0.3%, about 0.4%, about 0.5%, about 0.6%, about 0.7%, about 0.8%, about 0.9%, about 1%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 15%, about 16%, about 17%, about 18%, about 19%, about 20%, about 22%, about 24%, about 26%, about 28%, about 30%, about 32%, about 34%, about 36%, about 38%, about 40%, about 42%, about 44%, about 46%, about 48%, about 50%, about 60%, about 70%, about 80%, or about 90%, of its nanoparticle weight.

Nanoparticles may be prepared using a wide variety of methods known in the art. For example, nanoparticles can be formed by methods as nanoprecipitation, flow focusing fluidic channels, spray drying, single and double emulsion solvent evaporation, solvent extraction, phase separation, milling, microemulsion procedures, microfabrication, nanofabrication, sacrificial layers, simple and complex coacervation, and other methods well known to those of ordinary skill in the art. Alternatively or additionally, aqueous and organic solvent syntheses for monodisperse semiconductor, conductive, magnetic, organic, and other nanomaterials have been described (Pellegrino et al., 2005, Small, 1:48; Murray et al., 2000, Ann. Rev. Mat. Sci; 30:545; and Trindade et al., 2001, Chem. Mat., 13:3843). Additional methods have been described in the literature (see, e.g., Doubrow, Ed., “Microcapsules and Nanoparticles in Medicine and Pharmacy,” CRC Press, Boca Raton, 1992; Mathiowitz et al., 1987, J. Control. Release, 5:13; Mathiowitz et al., 1987, Reactive Polymers, 6:275; and Mathiowitz et al., 1988, J. Appl. Polymer Sci., 35:755; U.S. Pat. Nos. 5,578,325 and 6,007,845; P. Paolicelli et al., “Surface-modified PLGA-based Nanoparticles that can Efficiently Associate and Deliver Virus-like Particles” Nanomedicine. 5(6):843-853 (2010)). In some examples, the nanoparticles used herein are those described in International Publication No. WO2019/226612, which is incorporated herein by reference for all purposes.

In some examples, the method disclosed herein comprises administering to the subject a therapeutically effective amount of the composition disclosed herein and a nanoparticle. In some embodiments, the nanoparticle comprises an anti-ICAM antibody.

In some embodiments, the nanoparticle composition described herein may be in a dosage form. The dosage forms can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavernous, intrathecal, intravitreal, intracerebral, gingival, subgingival, intracerebroventricular, and intradermal. Such formulations may be prepared by any method known in the art.

The disclosed methods can be performed any time prior to or after RSV infection. In some aspects, the disclosed methods can be employed 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 years; 12, 11, 10, 9, 8, 7, 6, 5, 4, 3, 2, or 1 months; 30, 29, 28, 27, 26, 25, 24, 23, 22, 21, 20, 19, 18, 17, 16, 15, 14, 13, 12, 11, 10, 9, 8, 7, 6, 5, 4, or 3 days; 60, 48, 36, 30, 24, 18, 15, 12, 10, 9, 8, 7, 6, 5, 4, 3, or 2 hours prior to the infection; or 1, 2, 3, 4, 5, 6, 7, 8, 9, 10, 15, 20, 25, 30, 35, 40, 45, 50, 55, 60, 75, 90, 105, 120 minutes; 3, 4, 5, 6, 7, 8, 9, 10, 11, 12, 15, 18, 24, 30, 36, 48, 60 hours; 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 or more days; 4, 5, 6, 7, 8, 9, 10, 11, 12 or more months; 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 years after the infection.

Dosing frequency for the composition of any preceding aspect, includes, but is not limited to, at least once every 12 months, once every 11 months, once every 10 months, once every 9 months, once every 8 months, once every 7 months, once every 6 months, once every 5 months, once every 4 months, once every 3 months, once every two months, once every month; or at least once every three weeks, once every two weeks, once a week, twice a week, three times a week, four times a week, five times a week, six times a week, or daily. In some embodiment, the interval between each administration is less than about 4 months, less than about 3 months, less than about 2 months, less than about a month, less than about 3 weeks, less than about 2 weeks, or less than less than about a week, such as less than about any of 6, 5, 4, 3, 2, or 1 day. In some embodiment, the dosing frequency for the nanoparticle composition includes, but is not limited to, at least once a day, twice a day, or three times a day. In some embodiment, the interval between each administration is less than about 48 hours, 36 hours, 24 hours, 22 hours, 20 hours, 18 hours, 16 hours, 14 hours, 12 hours, 10 hours, 9 hours, 8 hours, or 7 hours. In some embodiment, the interval between each administration is less than about 24 hours, 22 hours, 20 hours, 18 hours, 16 hours, 14 hours, 12 hours, 10 hours, 9 hours, 8 hours, 7 hours, or 6 hours. In some embodiment, the interval between each administration is constant. For example, the administration can be carried out daily, every two days, every three days, every four days, every five days, or weekly Administration can also be continuous and adjusted to maintaining a level of the compound within any desired and specified range.

### EXAMPLES

The following examples are set forth below to illustrate the compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. RSV Infected Mice Display Altered Microbiota

To quantify bacterial phyla composition in RSV infection of female Balb/c mice, fecal material was collected every day during the study period, with genomic DNA isolated from the stool using QIAamp DNA stool minikit. Then, 16S rRNA gene-targeted group-specific primers designed and validated for quantification of the predominant bacterial species in mouse feces by real-time PCR.

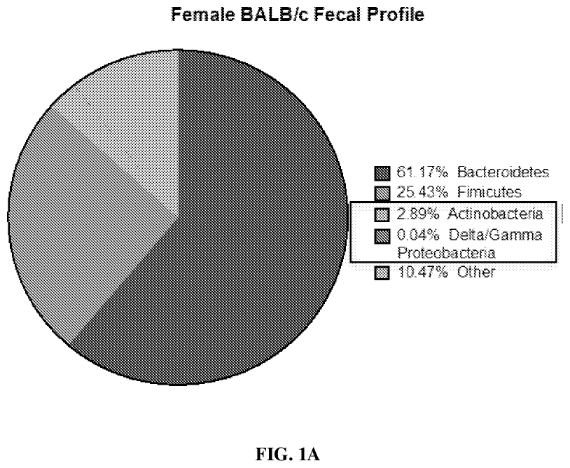

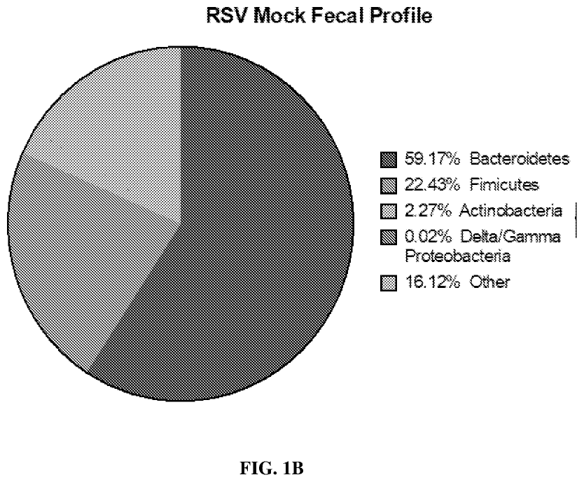

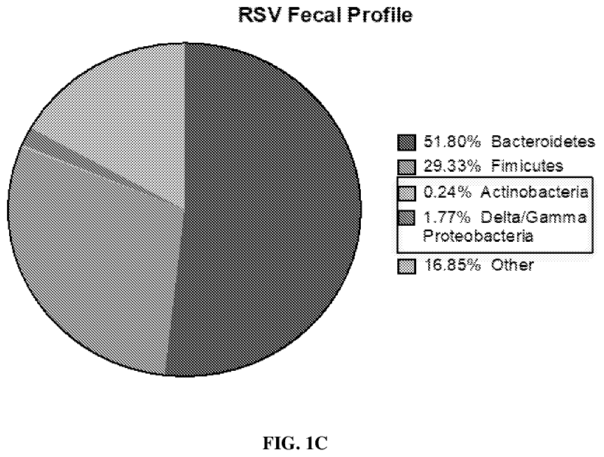

The results in FIGS. 1A-1C showed that compared to control mice, RSV infected mice showed about a significant decrease in Bacteroidetes (from 61% to 52%) and a 7% increase Firmicutes. Further, fecal samples from RSV infected mice showed a 10-fold decrease in Actinobacteria and a 60-fold increase D/G Proteobacteria.

### Example 2. Treatment with the Multifunctional Nanosystem Reverses Back the Gut Microbiota

To quantify bacterial phyla composition in RSV infection of female Balb/c mice, fecal material was collected every day during the study period, with genomic DNA isolated from the stool using QIAamp DNA stool minikit. Then, 16S rRNA gene-targeted group-specific primers designed and validated for quantification of the predominant bacterial species in mouse feces by real-time PCR.

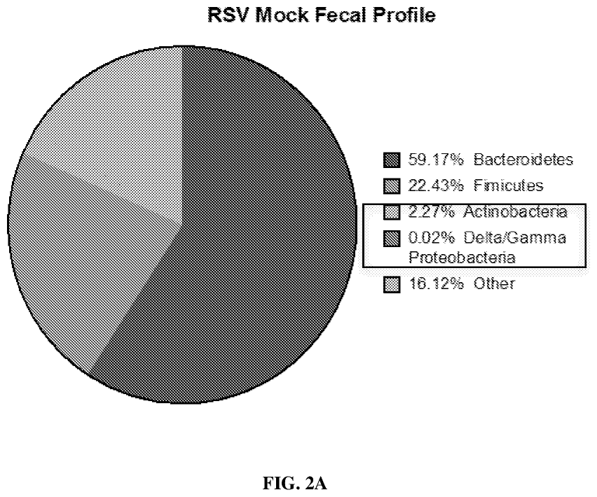

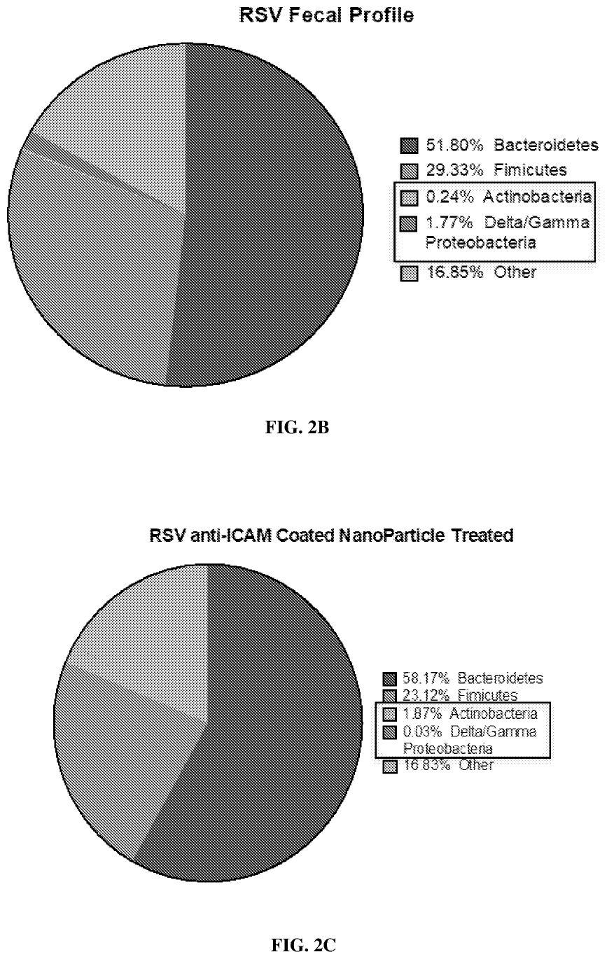

The results in FIGS. 2A-2C showed that compared to RSV-infected, Nanosystem-treated RSV infected mice showed about a significant increase in Bacteroidetes and a decrease Firmicutes. Further, fecal samples from RSV infected mice vs treated mice showed significant reversal showing increase in Actinobacteria and decrease D/G Proteobacteria.

### Example 3. Combination of Short-Chain Fatty Acids Significantly Affect RSV Infection

SCFAs are produced from the fermentation of undigestible soluble fiber by the intestinal microbiota, specifically Actinobacteria and Bacteroidetes populations. SCFAs have been shown by several groups to be a ligand for g-protein coupled receptors (GPCRs) such as Gpr41, Gpr43, and Gpr109a, as well as an inhibitor of histone deacetylases. The expression of these occurs on a wide range of cell types and modulate activities from metabolic homeostasis to immune system activity. Fiber induced attenuation of respiratory viral infection through SCFAs can be seen for RSV and Influenza. Also, mouse models supplemented with dietary oligosaccharides show an increased protective Th1 immune response against RSV challenge. Apart from infectious diseases, other studies show that SCFA and dietary fiber contribute to a protective effect against the development of allergic conditions and inflammatory disease, indicated that SCFA plays a role in immune homeostasis.

Metabolites of the gut microbiome that have immuno-modulatory as well as tissue anti-viral effects. For example, acetate, produced by bacteroidetes, can contribute acetyl units to lipogenesis in the cytosol of hepatocytes and adipocytes but its primary site of oxidation is peripheral muscle. Also, Butyrate, produced by Firmicutes bacteria, is largely oxidized at the gut epithelium where it has been implicated in orchestrating the tight junction protein complexes to control gut barrier function, regulating inflammatory cell populations (inhibits NF-kB signaling) and functioning through receptor-mediated and histone deacetylation mechanisms. Further, Propionate, produced by bacteroidetes, can act locally in the gut on enteroendocrine L-cells to stimulate release of the anorexigenic gut hormones. While these actions of metabolites were not indicative of preventing RSV induced lung disease, because of the microbiota changes, their role in RSV-infected lung epithelial cells was tested using A549 as a model.

To examine effects of these SCFAs on RSV infection, a recombinant RSV-L19F variant that expressed red fluorescent protein (RFP) was utilized and the expression of RFP was used to determine cell infection. RFP was normalized to nuclear stain DAPI, giving an RFP/DAPI value. Twenty-four hours after plating, the media was removed, and the cells were infected at 3 MOI with RFP expressing RSV-L19F for 3 hours in 75 uL of OptiMEM media. The infectious inoculum was then removed and replaced with the SCFA media administered in the same manner as previously explained. Cells were incubated for 48 hours, then stained with DAPI, and imaged with Keyence. ImageJ was used to analyze and quantify the fluorescence.

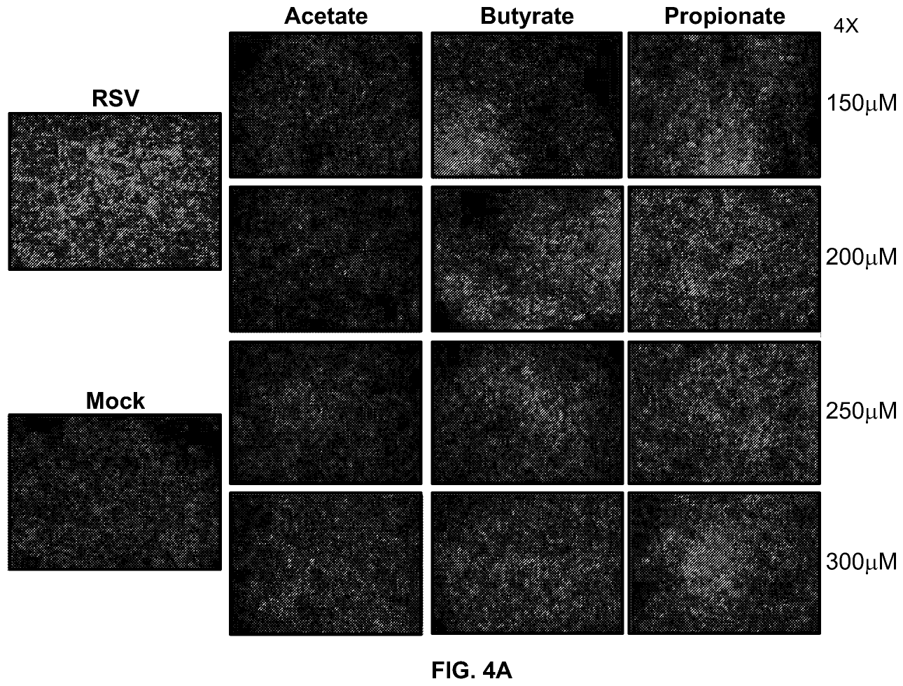

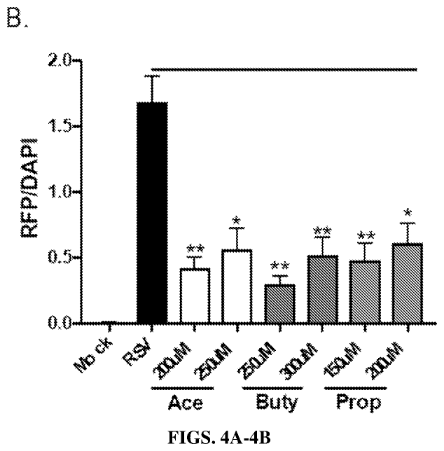

To test the effect of these SCFAs on viral infection, A549 cells were plated in a 96 well plate and concentrations ranging from 100 to 300 μM in triplicate of acetate, butyrate, and propionate were tested 24 hours before RSV infection (FIGS. 4A-4B). Cells were infected at 3 MOI of RSV Line 19 (rA2-KL19F) expressing a red fluorescent marker, mKate2, as well as the F protein from the mucogenic clinical strain, and media was replaced with the appropriate SCFAs that was previously used in cultured. After 48 hours, the cells were stained with NucBlue (life technologies) and imaged using a Keyence fluorescence microscope (FIGS. 4A-4B). The results show that treatment with acetate, butyrate or propionate reduces the expression of RFP-MKate in this experiment. Additionally, to rule out any significant changes in pH that might contribute to these findings, normal media was compared with three samples of media with 1 mM each of respective SCFA added and saw no change in the pH of the media. These results show that each of these SCFAs does inhibit viral replication in A549 cells.

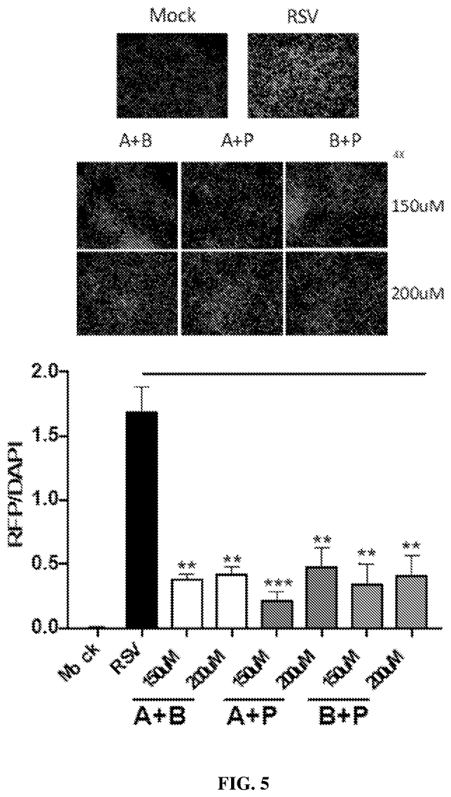

While all three SCFAs tested showed a reduction of viral infection, the individual SCFAs use distinct receptors and butyrate is known to have anti-inflammatory activity. Therefore, whether combinations of acetate (A), propionate (P), and butyrate (B) can have additive or synergistic effects was tested. Thus, concentrations of SCFAs used in culture were taken from the preliminary data and SCFAs were combined for treating a viral infection in vitro (FIG. 5). The results showed that these combinations of acetate+butyrate (A+B), acetate+propionate (A+P), and butyrate+propionate (B+P) at a range of 150-200 μM significantly reduced RFP expression from the rA2 K19L-mKate strain. These data show that the combinations of SCFAs can be used to combat viral infection.

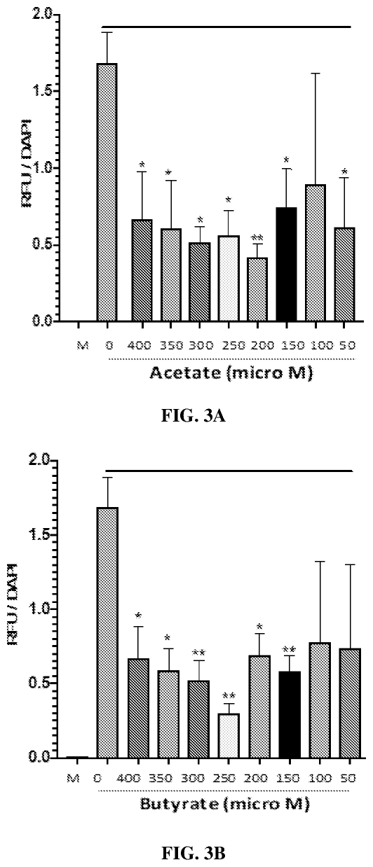

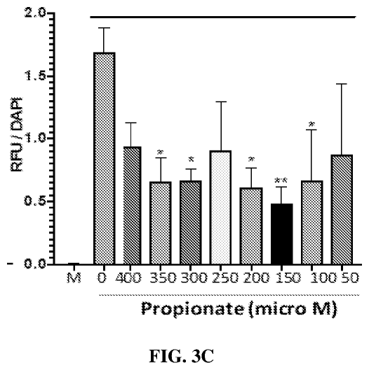

The results in FIGS. 3A-3C showed that these SCFAs reduced the RSV infection of A549 cells, at high doses in a dose-dependent fashion in the range of 150 μM to 400 μM, but not at low doses. At a dose lower than 150 μM, RSV infection was still inhibited.

### Example 4. Combination of Short-Chain Fatty Acids Significantly Affect RSV Replication

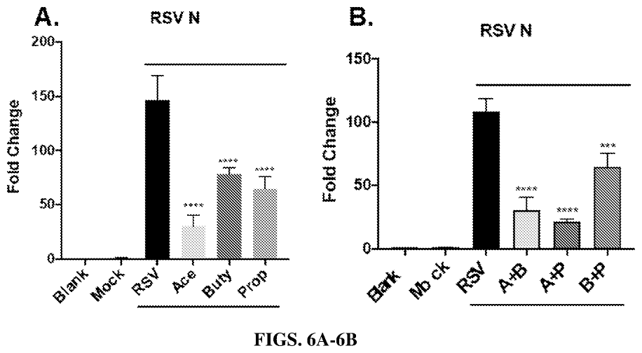

To examine effects of the combination of SCFAs on RSV replication, a recombinant RSV-L19F variant that expressed red fluorescent protein (RFP) was utilized and the expression of RFP to determine cell infection was used. RFP was normalized to nuclear stain DAPI, giving an RFP/DAPI value. Twenty-four hours after plating, the media was removed, and the cells were infected at 3 MOI with RFP expressing RSV-L19F for 3 hours in 75 uL of OptiMEM media. The infectious inoculum was then removed and replaced with the SCFA media administered in the same manner as previously explained. Cells were incubated for 48 hours, then stained with DAPI, and imaged with Keyence. ImageJ was used to analyze and quantify the fluorescence. The RNA was isolated from these cells, subjected q PCR for RSV N gene expression reduced of RSV N as determined by qPCR in A549 cells. The results of RSV gene expression are shown in FIGS. 6A-6B.

### Example 5. SCFAs and Combinations Promote IFNβ Production

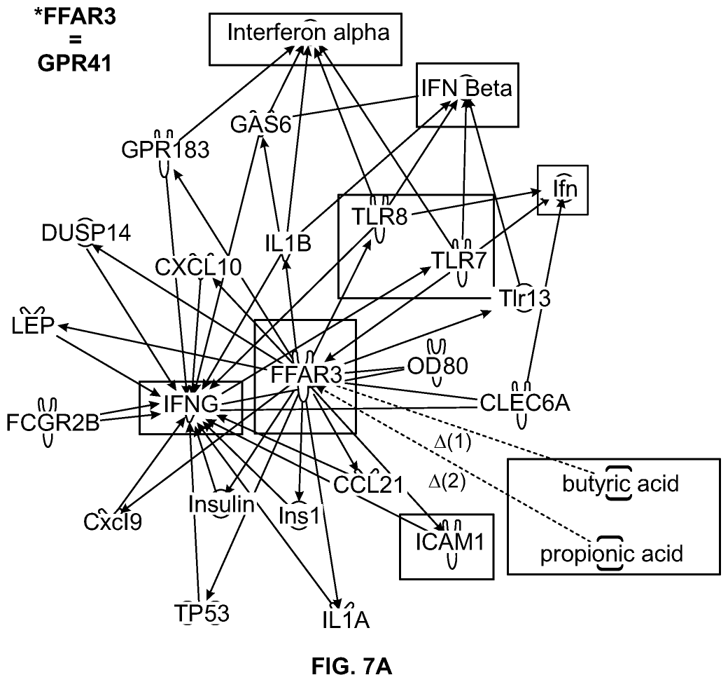

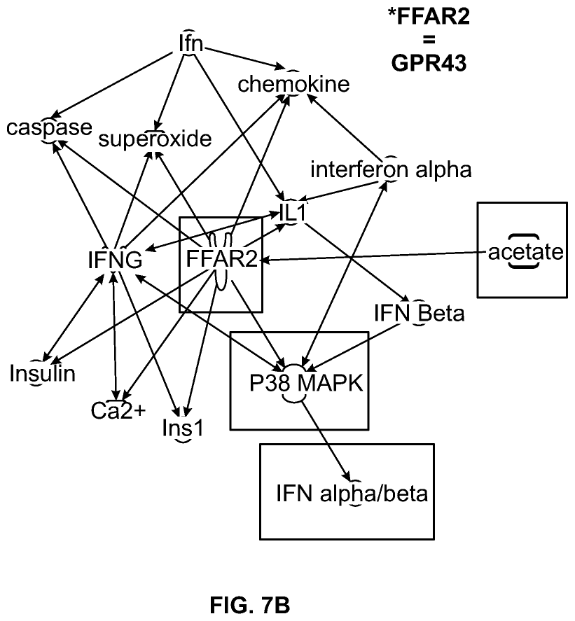

To gain a better understanding of the phenotypic changes that could occur from SCFA signaling, a network analysis was performed on FFAR 2/3 activation by SCFA ligands, which were generated through the use of Ingenuity Pathway Analysis (IPA) (QIAGEN Inc.) (FIGS. 7A-7B). This analysis shows that the SCFAs can have conserved upregulation of specified cytokines, such as Type-1 interferons, which are known to be antiviral, as well as metabolic implications. Thus, acetate is the ligand for FFAR2, whereas butyrate and propionate are the ligands for FFAR3, which show upregulation of cytokines, interferons, and toll-like receptors; thus showing a benefit from novel combinations of microbiome-inspired metabolites to maximize therapeutic efficacy in combating viral infections and disease. A549 cells possess GPR41 and 43 receptors, which as indicated by the IPA analysis upregulates the production of inflammatory cytokines when stimulated by SCFAs.

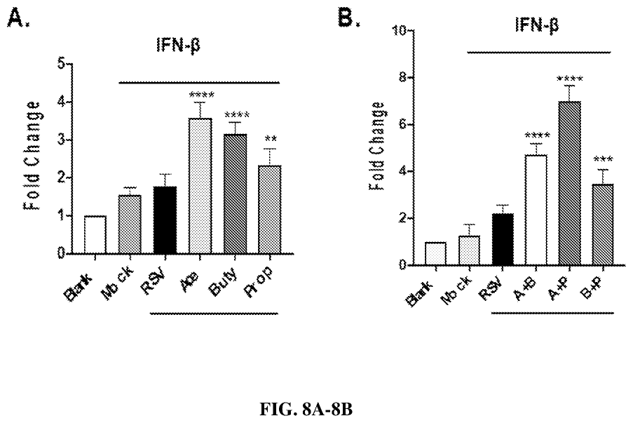

RSV is known to antagonize type-1 interferon signaling by the action of its non-structural proteins (NS1 and NS2), so activation of interferon by SCFAs can be a driving force behind the observed antiviral effect. Data in FIGS. 7A-7B shows that the SCFAs upregulate the production of IFN-β, an anti-viral type 1 interferon, during RSV infection. This effect is seen in both individually administered SCFAs, as well as in combination (FIGS. 8A-8B). These findings are also in agreement with the findings of the IPA analysis (FIGS. 7A-7B) and the reduction of RSV seen in FIGS. 6A-6B.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A method for preventing or treating respiratory syncytial virus (RSV) infection in a subject, comprising administering to the subject a therapeutically effective amount of a composition comprising a short-chain fatty acid.

2. The method of claim 1, wherein the short-chain fatty acid is produced by Bacteroidetes or Fimicutes.

3. The method of claim 1, wherein the short-chain fatty acid is produced by Bacteroidetes.

4. The method of claim 1, wherein the short-chain fatty acid is produced by Fimicutes.

5. The method of claim 1, wherein the short-chain fatty acid comprises acetate, butyrate, or propionate or a combination thereof.

6. The method of claim 1, wherein the short-chain fatty acid comprises acetate.

7. The method of claim 1, wherein the short-chain fatty acid comprises butyrate.

8. The method of claim 1, wherein the short-chain fatty acid comprises propionate.

9. The method of claim 1, wherein the short-chain fatty acid comprises acetate and butyrate.

10. The method of claim 1, wherein the short-chain fatty acid comprises acetate and propionate.

11. The method of claim 1, wherein the short-chain fatty acid comprises butyrate and propionate.

12. The method of claim 1, wherein the short-chain fatty acid is encapsulated within a nanoparticle.

13. A composition for preventing or treating respiratory syncytial virus (RSV) infection, wherein the composition comprises acetate and butyrate, acetate and propionate, or butyrate and propionate.

14. The composition of claim 13, wherein the composition comprises acetate and butyrate.

15. The composition of claim 13, wherein the composition comprises acetate and propionate.

16. The composition of claim 13, wherein the composition comprises butyrate and propionate.
